(19) United States
(12) Patent Application Publication
(10) Pub. No.: US 2017/0342213 A1
(43) Pub. Date: Nov. 30, 2017

(54) ELECTRICALLY CONDUCTIVE POLYMERIC MATERIAL

(71) Applicant: Newsouth Innovations PTY Limited, Sydney (AU)

(72) Inventors: Rylie Adelle Green, Marrickville (AU); Laura Anne Poole-Warren, Coogee (AU); Josef Goding, Marrickville (AU); Alexander Patton, Fraser (AU)

(21) Appl. No.: 15/538,281

(22) PCT Filed: Dec. 24, 2015
(86) PCT No.: PCT/AU2015/050846

§ 371 Date: Jun. 21, 2017

(30) Foreign Application Priority Data
Dec. 24, 2014 (AU) 2014905282

(51) Int. Cl. C08G 73/02; C08L 83/04; C08L 33/06; C08L 41/00; C08L 75/04; H01B 1/12; C08L 39/04; C08L 79/02; A61N 1/05; C08F 222/10

(52) U.S. Cl. CPC C08G 73/0266; H01B 1/124; C08L 83/04; C08L 33/06; C08L 39/04; C08L 41/00; C08L 75/04; A61N 1/05; C08G 2261/51; C08G 2261/44; C08F 2222/104; C08G 2261/1424; C08G 2261/3223; C08L 79/02

(57) ABSTRACT

The invention provides a method of preparing an electrically conductive polymeric material. The method comprises providing a polymeric network having a short chain conductive polymer dispersed in the polymeric network and electropolymerising a conductive polymer within the polymeric network. Also described is a free standing flexible electrically conductive polymeric material comprising a conductive polymer within a polymeric network.

### FIELD OF THE INVENTION

The present invention relates to electrically conductive polymeric materials, and to a method of preparing an electrically conductive polymeric material.

### BACKGROUND

Conductive polymers are polymers that are able to conduct electricity. Conductive polymers have a variety of applications.

For example, conductive polymers have been used in bioelectronic devices. Historically, metals have been used to interface with the excitable tissues of the body (e.g. nerves, cardiac tissue and skeletal muscle), to inject charge or record tissue activity. Conventional metal electrodes are usually fabricated from platinum (Pt) or Pt alloys but these materials have limitations including a relatively low charge injection limit, high stiffness and poor biorecognition. However, with the miniaturization of electronics, the need for smaller implantable electrode arrays, which can target cells with high selectivity, has driven the development new electrode material technologies. Materials including conductive polymers (CPs) and conductive hydrogels (CHs) have been used to create organic bioelectronic electrode coatings. While these coatings have been shown to improve the performance of metallic electrodes, the development of soft and flexible arrays has been limited by the need for the underlying metal array, which imparts increased stiffness and fabrication limitations.

Forming soft, flexible biocompatible electrodes is desirable for bionic devices and brain-machine interfaces. Despite CPs being softer than their metallic counterparts, their brittle mechanical properties and friable surface characteristics have limited their use.

Growth of CPs by electropolymerisation (also referred to as electrodeposition) typically occurs from nucleation sites at a metallic electrode interface, which has seen them used for coating medical electrodes to improve charge injection capacity. During a typical polymerisation the CP monomer is oxidised under a positive voltage, the amplitude of which is dependent on the monomer, dopant and electrolyte choice, and forms oligomers which precipitate out of solution when the chain reaches a critical length. The oxidation potential for polymerisation is lowest at the electrode surface and as a result, the CP precipitates at the electrode surface where free radicals are generated and hence nucleation occurs. This is known as primary spontaneous nucleation. This mechanism of polymerisation generally leads to compact growth of the CP on an electrode surface. While CPs produced in this manner tend to have superior electrical properties compared with conventional metallic electrodes, they often suffer from delamination or mechanical failure as they are brittle and friable.

Recent studies have determined that such electropolymerisation techniques can be used to grow CPs within hydrogels to produce conductive hydrogels (CHs). CHs are softer, tissue-like conductive materials that have broad utility in tissue engineering for electro-excitable organs including implantable electrodes, nerve guides and cardiac patches. Formation of CHs can be achieved by providing covalently bound anionic dopants as part of the hydrogel mesh, which encourages growth and precipitation of CP chains within the hydrogel. However, disadvantages of such systems include the requirement for a conductive substrate that remains tightly bound to the soft coating and limits to the thickness of the CHs which can be produced. Electropolymerisation from a bulk metal electrode physically binds the hydrogel to the underlying electrode as the highly nodular CP mechanically interlocks with imperfections on the electrode surface before growing through the hydrogel. This limits flexibility and ease of fabrication since the bound underlying electrode, which is often a stiff metallic substrate such as platinum or indium tin oxide coated glass, must be removed. Additionally, when hydrogel thickness exceeds 100-200 μm, penetration of the CP through the hydrogel is restricted, hindering the formation of interpenetrating networks of the two polymer systems.

Recently, soft CHs with a charge injection limit that is up to 10 times greater than Pt and a stiffness moduli which is more than 3 orders of magnitude lower have been produced. These materials were produced from a composite of polyvinyl alcohol (PVA) crosslinked with heparin, to form an anionic hydrogel, through which poly(3,4-ethylene dioxythiophene) (PEDOT) was grown. However, the electropolymerization method used to grow the CP within the hydrogel is performed using a metallic substrate (Pt, gold or indium tin oxide), which inevitably becomes bound to the CP and also limits the growth of the CP to less than 50 μm thick.

It would therefore be advantageous to provide an alternative method for forming electrically conductive polymeric materials.

### SUMMARY OF THE INVENTION

In a first aspect, the present invention provides a method of preparing an electrically conductive polymeric material, the method comprising:
providing a polymeric network having a short chain conductive polymer (SCCP) dispersed in the polymeric network;
electropolymerising a conductive polymer (CP) within the polymeric network.

In the method of the present invention, the short chain conductive polymer provides a nucleation site for the electropolymerisation of the conductive polymer.

In one embodiment, the polymeric network is a hydrogel.

In another embodiment, the polymeric network is an elastomer.

In one embodiment, the polymeric network, prior to electropolymerisation of the conductive polymer, is non-conductive.

In one embodiment, the short chain conductive polymer has from about 5 to about 1000 monomeric units.

In one embodiment, the short chain conductive polymer is poly(3,4-ethylene dioxythiophene)-poly(styrene sulfonate) (PEDOT:PSS) or tetramethacrylate poly(3,4-ethylene dioxythiophene).

In one embodiment, the conductive polymer is PEDOT, polypyrrole or polyaniline.

In one embodiment, the electropolymerisation of the conductive polymer comprises:
contacting the polymeric network with a solution comprising monomer of the conductive polymer; and
applying an electrical potential across the polymeric network.

In one embodiment, the polymeric network having a SCCP dispersed in the network comprises a localised region of a polymeric material.

In one embodiment, the electrically conductive polymeric material has a conductivity of greater than about 10 S/cm.

In one embodiment, the electrically conductive polymeric material has a charge storage capacity of greater than about 10 mC/cm².

In a second aspect, the present invention provides a device comprising an electrically conductive polymeric material prepared by the method of the first aspect.

In a third aspect, the present invention provides a free standing flexible electrically conductive polymeric material comprising a conductive polymer within a polymeric network.

In one embodiment, the conductive polymer is present in the polymeric network in the form of a non-particulate dispersion.

In one embodiment, the polymeric network is a hydrogel.

In one embodiment, the polymeric network is an elastomer.

In one embodiment, the conductive polymer is PEDOT, polypyrrole or polyaniline.

In one embodiment, the conductive polymeric material has a conductivity of greater than about 10 S/cm.

In one embodiment, the conductive polymeric material has a charge injection limit of more than 300 μC/cm.

In one embodiment, the conductive polymeric material has a dimension of greater than 200 μm in all directions.

In a fourth aspect, the present invention provides a polymeric material comprising one or more regions which are electrically conductive and one or more regions which are non-conductive, wherein the conductive regions and non-conductive regions are integrally bound to each other and wherein at least one of the electrically conductive regions has a dimension of greater than about 200 μm in all directions.

### BRIEF DESCRIPTION OF THE FIGURES

Preferred embodiments of the present invention are described below, by way of example only, with reference to the accompanying drawings in which:

FIG. 1 shows a tree diagram depicting three mechanisms of nucleation for conductive polymer growth within hydrogels by electrochemical polymerisation.

FIG. 2 shows photographic images of bulk metallic glass (Mg₆₄Zn₃₀Ca₅Na₁; BMG) loaded PVA at: A. 5 wt %, B. 10 wt % and C. 15 wt % from Example 1. (Scale bar=5 mm)

FIG. 3 shows photographic images of PVA loaded with PEDOT:PSS at: A. 0.01 wt %; B. 0.05 wt %; C. 0.1 wt %; and D. 0.5 wt % from Example 1. (Scale bar=5 mm)

FIG. 4 shows a graphical representation of the charge storage capacity (CSC) of hydrogels loaded with: A. BMG particles; and B. PEDOT:PSS from Example 1. (Individual data shown with mean (centre line); Error bars represent 1 standard deviation (n=6))

FIG. 5 shows graphical representations from Example 1 of: A. charge storage capacity; and B. electrochemical impedance of 10 wt % BMG loaded PVA-Hep after 80 mins of electropolymerisation (Deposition Time). (Electrochemical measurements were made in a three-electrode cell with voltage applied to a stainless steel substrate on which the hydrogel disc was placed in a DPBS solution with a platinum counter electrode and an isolated Ag/AgCl reference electrode; Mean values are shown and error bars represent 1 standard deviation (n=6))

FIG. 6 shows photographic images of BMG loaded PVA-Hep after 80 mins of PEDOT electropolymerisation from Example 1 at: A. low magnification (100×); and B. high magnification (400×).

FIG. 7 shows graphical representations from Example 1 of: A. charge storage capacity; and B. impedance of PEDOT:PSS loaded PVA following electropolymerisation of PEDOT for 80 mins at 0.5 mC/cm². (Electrochemical measurements were made in a three-electrode cell with voltage applied to a stainless steel substrate on which the hydrogel disc was placed in a DPBS solution with a platinum counter electrode and an isolated Ag/AgCl reference electrode; Mean values are shown and error bars represent 1 standard deviation (n=6); *=statistical significance with unpaired t-test (p<0.01)).

FIG. 8 shows light microscope images from Example 1 taken at 100× magnification of PEDOT:PSS loaded PVA after 10, 20, 40 and 80 mins of PEDOT electropolymerisation. The PEDOT:PSS was incorporated at 0.01, 0.05, 0.1 and 0.5 wt %.

FIG. 9 shows graphical representations from Example 1 of: A. CV hysteresis curves for the 0.5 wt % PEDOT:PSS loaded PVA; and B. the corresponding CSC (n=6) for the 0.1 wt % and 0.5 wt % PEDOT:PSS loaded PVA with up to 160 mins of PEDOT electropolymerisation (Deposition Time). (*=statistical significance with unpaired t-test (p<0.01))

FIG. 10 shows graphical representations from Example 1 of impedance magnitude and phase lag over 160 mins of PEDOT electropolymerisation for: A. 0.1 wt %; and B. 0.5 wt % PEDOT:PSS loaded PVA. (Data represents the mean and one standard deviation (n=6))

FIG. 11 is a schematic depiction showing the fabrication of conductive hydrogel tracks within a non-conductive hydrogel as described in Example 2.

FIG. 12 shows photographic images of the patterning of the hydrogel of Example 2 by silicone mould wherein: A. shows a top view; and B. shows a side view of the construct after step 3, before electropolymerisation of PEDOT.

FIG. 13 shows optical microscopy images at 400× magnification of the conductive hydrogel track showing progression of PEDOT growth at: A. 0 min; B. 10 min; and C. 20 min post-electropolymerisation from Example 2.

FIG. 14 is a graphical representation of the cyclic voltammetry curves from Example 2 showing the increased charge transfer from the formation of PEDOT within the CH track.

FIG. 15 shows phase contrast images from Example 2 of HL-1 cell proliferation on: A. TCP; B. CH track before electropolymerisation; and C. CH track after electropolymerisation of PEDOT for 20 min.

### DETAILED DESCRIPTION OF EMBODIMENTS OF THE INVENTION

In a first aspect, the present invention provides a method of preparing an electrically conductive polymeric material, the method comprising:
providing a polymeric network having a short chain conductive polymer (SCCP) dispersed in the polymeric network; and
electropolymerising a conductive polymer (CP) within the polymeric network.

In the method of the present invention, the short chain conductive polymer (SCCP) provides a nucleation site for the electropolymerisation of the conductive polymer (CP).

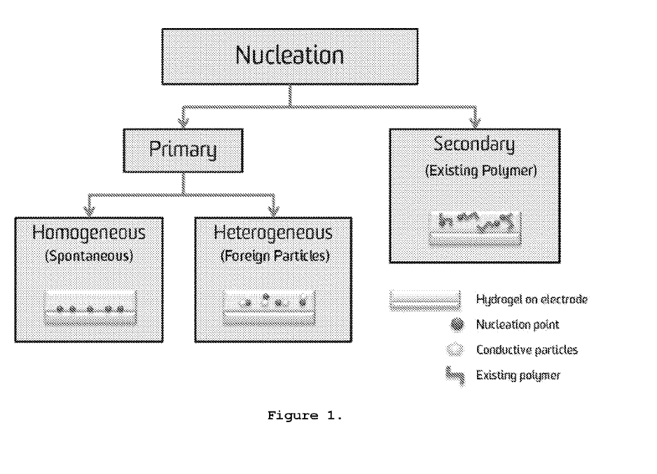

The inventors have surprisingly found that a short chain conductive polymer dispersed in a polymeric network can act as a nucleation site for the electropolymerisation of a conductive polymer within the polymeric network, enabling the electropolymerisation of a conductive polymer within the polymeric network. The inclusion of the SCCP dispersed in the polymeric network enables the electropolymerisation of the conductive polymer throughout the polymeric material. In the method of the invention, nucleation for the growth of the conductive polymer occurs due to secondary mechanisms, as distinct from the primary mechanisms (both shown in FIG. 1), and these are believed to occur according to the Gibbs free energy principle, where the chemical potential is minimised. Whereas previous methods to form conducting polymers within polymeric networks resulted in nucleation and polymer chain growth from the site of an electrode (primary nucleation), the method disclosed herein provides nucleation sites, in the form of SCCPs, that are dispersed throughout the polymeric network. These nucleation sites may be described as secondary nucleation sites (see FIG. 1).

The method of the present invention can be used to prepare freestanding electrically conductive polymeric materials, that is, electrically conductive polymeric materials that are not bound to an inorganic surface, such as a rigid metal surface. Advantageously, the method of the invention can be used to prepare electrically conductive polymeric materials that are soft, flexible and/or deformable.

The method of the invention can also be used to prepare polymeric materials having a pattern of conductive regions and non-conductive regions. The conductive regions can be prepared by the method of the invention without lamination on, or being grown up from, a conductive base such as a metal surface.

### Polymeric Network

The polymeric network may be any polymeric network. Preferably the polymeric network is swellable in a solvent. A polymeric network that is swellable in a solvent is preferred as the swelling of the network can facilitate the introduction of polymer subunits capable of forming the conductive polymer (e.g. a monomer capable of forming the conductive polymer) throughout the polymeric network prior to the electropolymerisation of the conductive polymer.

In a preferred embodiment, the polymeric network is a hydrogel. In another embodiment, the polymeric network is an elastomer, such as a polyurethane elastomer or a silicone rubber elastomer. In some embodiments, the hydrogel or elastomer may comprise two or more polymer constituents in order to take advantage of the properties that each of the polymer constituents impart to the resultant hydrogel or elastomer.

Non-limiting examples of polymers suitable for forming a hydrogel or elastomer to provide the polymeric network include polyvinyl alcohol (PVA), polyethylene glycol, poly(acrylic acid) and its derivatives; poly(ethylene oxide) and its copolymers, polyphosphazene, silicones, polyacrylamides, polyvinylpyrrolidones, poly-hydroxy ethylmethacrylate, poly(styrene sulfonate), polyurethanes and its derivatives; or combinations thereof.

The polymeric network may be formed by methods known in the art for preparing polymeric networks.

For example, a hydrogel may be formed by mixing one or more polymer subunits capable of forming a hydrogel and subjecting the mixture to conditions suitable for polymerising and cross-linking the polymer subunits to form a cross-linked polymer. As used herein, the term “polymer subunit” refers a monomer, dimer, macromer (e.g. oligomer) or mixture thereof, that, upon polymerisation, forms a polymer. As the person skilled in the art will appreciate, the methods used to promote polymerisation and cross-linking of the polymer subunits to form the cross-linked polymer will depend on the polymer subunit or polymer subunits used. Suitable conditions for different polymer subunits can be readily determined by a person skilled in the art. In some embodiments, the polymerisation and cross-linking reaction is a radical polymerisation reaction. Radical polymerisation reactions may be initiated by a variety of techniques, including, for example, by use of a chemical initiator, exposure to UV light or exposure to visible light in the presence of a photocatalyst. For example, to form a poly(vinyl alcohol) methacrylate (PVA-MA) hydrogel, a 20 wt % PVA-MA macromer solution may be photopolymerized by exposure to UV light (for example 30 mW/cm², 365 nm for 180 s) to promote cross-linking (polymerisation). A similar method can be used to prepare polyethylene glycol (PEG) hydrogels. As a further example, a polyethylene glycol (PEG) hydrogel may also be formed by forming a 15 wt % PEG-tyramine macromer solution and photopolymerising the macromer solution by exposure to visible light in the presence of a persulfate salt and a ruthenium catalyst. Other methods and other polymer subunits would be known to those skilled in the art and a person skilled in the art will readily be able to determine appropriate methods for preparing a polymeric network.

The SCCP may be incorporated in the polymeric network by any means that results in the SCCP being dispersed in part or all of the polymeric network. Typically the SCCP is incorporated in the polymeric network during the formation of the polymeric network.

For example, when the polymeric network is a hydrogel, the SCCP is typically dispersed in the mixture of the polymer subunits used to form the hydrogel prior to the polymerisation and cross-linking of the polymer subunits to form the hydrogel. For example, to form a PVA-MA hydrogel comprising the SCCP poly(3,4-ethylene dioxythiophene)-poly(styrene sulfonate) (PEDOT:PSS), PEDOT:PSS may be dispersed within a 20 wt % PVA-MA macromer solution in an amount of about 0.01 to about 1 wt %, e.g. about 0.1 to 0.5 wt % or 0.1 to 1 wt %, and the solution photopolymerized by exposure to UV light (for example 30 mW/cm², 365 nm for 180 s) to promote cross-linking (polymerisation) of the PVA-MA macromer, producing a PVA-MA hydrogel comprising PEDOT:PSS dispersed in the hydrogel.

Similarly, when the polymeric network is an elastomer, the SCCP is typically dispersed in the mixture used to form the elastomer prior to curing of the mixture to form the elastomer.

In some embodiments, the method of the present invention comprises a step, prior to the electropolymerisation of the conductive polymer, of preparing the polymeric network having a short chain conductive polymer dispersed in the polymeric network. This step may comprise preparing a mixture comprising a short chain conductive polymer and polymer subunits capable of forming a polymeric network (e.g. by mixing a short chain conductive polymer and one or more polymer subunits capable of forming a polymeric network), and exposing the mixture to conditions whereby the polymer subunits polymerise to form a polymeric network having the short chain conductive polymer dispersed in the polymeric network.

Accordingly, in some embodiments, the method of the first aspect of the present invention comprises the steps of:
providing a mixture of a short chain conductive polymer and one or more polymer subunits capable of forming a polymeric network;
exposing the mixture to conditions whereby the polymer subunits polymerise to form a polymeric network having the short chain conductive polymer dispersed in the polymeric network; and
electropolymerising a conductive polymer (CP) within the polymeric network.

Typically, the mixture comprises a solution or dispersion of the short chain conductive polymer and the one or more polymer subunits capable of forming a polymeric network in a solvent or carrier. Advantageously, in some embodiments, the short chain conductive polymer and the one or more polymer subunits capable of forming a polymeric network may be in an aqueous solution.

The short chain conductive polymer (SCCP) is preferably immobilised within the polymeric network. In some embodiments, the SCCP is entangled with the polymer constituents of the polymeric network. In other embodiments, the SCCP is covalently bound to the polymer constituents of the polymeric network.

In a preferred embodiment, the polymeric network, prior to the electropolymerisation of the conductive polymer, is non-conductive. As used herein, the term “non-conductive” refers to a resistance of greater than about 1 Megaohm/cm.

Preferably, the polymeric network is not bound to the surface of an electrode.

### Short Chain Conductive Polymer (SCCP)

In the method of the present invention, the SCCP provides a nucleation site for the formation of the conductive polymer. The SCCP may be any short chain conductive polymer. There are many SCCPs, both commercially available and otherwise, that are suitable for use in the method. Typically, the SCCP is no more than 10000 monomeric units in length. In some embodiments, the SCCP is no more than 1000 monomeric units in length. In some embodiments, the SCCP used in the method has from about 5 to about 1000 monomeric units. In some embodiments, the SCCP comprises from about 5-800, 5-500, 5-100, 5-80, 5-50, 5-25, 5-10, 10-1000, 10-800, 10-500, 10-100, 10-80, 10-50, 10-25, 20-1000, 20-800, 20-500, 20-100, 20-80, or 20-50 monomeric units. In some embodiments, the backbone of the SCCP comprises less than about 3000 atoms, for example, less than 1000 or less than 500 atoms. The SCCP is typically formed by chemical polymerisation to control the chain length and properties of the SCCP.

In the method disclosed herein, the SCCP is dispersed in the polymeric network. This dispersion is typically uniform, but there is no requirement for the dispersion to be uniform. In some embodiments, the SCCP is unevenly dispersed throughout the polymeric network leading to regions having an increased concentration of the SCCP and other regions having a decreased concentration of SCCP. In some embodiments this may be used to provide a pattern within the polymeric network that is then used to provide regions for nucleation to take place to form a pattern of conductive polymer within the polymeric network. In other embodiments the non-uniform (i.e. variable) dispersion may be used to fine-tune the formation of conductive polymer and hence the conductive properties of the resultant electrically conductive polymeric material. In other words, the dispersion of the SCCP can be used to control the formation of the conductive polymer within the polymeric network. In other embodiments there is a uniform dispersion of the SCCP in the polymeric network.

The SCCP may, for example, be included in the polymeric network in concentrations of about 0.005 to 24 wt % relative to the total weight of the polymeric network, although, as a person skilled in the art will appreciate, this will depend on both the polymeric network as well as the SCCP that are employed. In some embodiments, the SCCP is included in the polymeric network in a concentration of about 0.005 to 5, 0.005 to 2, 0.01 to 2, 0.05 to 2, 0.1 to 2, 0.005 to 1, 0.01 to 1, 0.05 to 1, 0.1 to 1, 0.005 to 0.5, 0.01 to 0.5, 0.05 to 0.5, or 0.1 to 0.5, wt % relative to the total weight of the polymeric network. Typically, the SCCP is included in the polymeric network is an amount less than that which would result in the resistance of polymeric network containing the SCCP being less than about 1 Megaohm/cm.

Non-limiting examples of SCCPs include short chain conductive polymers formed of polypyrrole or its derivatives, polythiophene or its derivatives, polyphenylene sulphide (i.e. a polymer formed from phenyl mercaptan) or its derivatives, polyaniline or its derivatives, polyindole or its derivatives, polycarbazole or its derivatives, polyacetylene or its derivatives or copolymers or combinations thereof. Preferred SCCPs include PEDOT:PSS and tetramethacrylate poly(3,4-ethylene dioxythiophene). An example of an SCCP is the product Orgacon made by AGFA Specialty Products.

### Dopant

As those skilled in the art will appreciate, a conductive polymer requires a dopant (e.g. an ionically charged species) in order for the polymer to form highly conductive pathways and be capable of passing electronic or ionic charges. Such dopants are typically sulfonated molecules (e.g. p-toluene sulfonic acid, poly(styrene sulfonate), dodecyl benzene sulfonate), but can be other groups such as perchlorates, carbonates or amino acids.

In the method of the present invention, a dopant is preferably present in the polymeric network. Preferably the dopant is immobilised within the polymeric network. For example, the dopant may form part of the polymer constituents of the polymeric network. Alternatively, the dopant may be bound to the SCCP which is covalently bound to, or entangled with, the polymer constituents of the polymeric network.

In some embodiments, the dopant is part of the SCCP. For example, in PEDOT:PSS the sulfonate group of the PSS provides the dopant in the form of the sulfonate anion covalently bound to the phenyl group of the polystyrene.

In one embodiment, the dopant is an anionic species covalently bound to the polymeric network or the SCCP. The polymer constituent having covalently bound anionic species may be a polymer that inherently contains an anionic charge in its backbone, or may be a polymer that has been modified to include a covalently bound anionic species. For example, polymer constituents such as DNA, heparin, alginate and chondroitin sulphate contain anionic species in their polymer backbones. Synthetic polymers or biopolymers such as peptides, proteins or saccharides having a specific bioactivity can be anionically modified using methods known in the art. For example, biopolymers can be functionalised by chemically modifying their end groups to create an overall anionic charge. For example laminin peptides can be modified by the addition of specific amino acids which create an anionic tail or side chain that would allow it to dope a conductive polymer whilst retaining its bioactivity.

As used herein, the term “biopolymer” refers to a polymer (e.g. a protein, peptide or saccharide) produced by a living organism or a synthetically produced mimic of a polymer produced by a living organism which has similar properties and activity when placed in a biological environment.

Typically the dopant is present in the polymeric network in an amount such that, after the electropolymerisation of the conductive polymer, the dopant is present in an amount 0.2 to 0.5 dopant per monomer of the conductive polymer. Such an amount of dopant facilities the formation of long chain conducting polymers and the formation of a highly conductive polymeric material.

### Conductive Polymer

A conductive polymer is a polymer which is able to conduct electricity. Conductive polymers are unsaturated polymers containing delocalised electrons. Conductive polymers typically comprise alternating saturated and unsaturated bonds in the backbone of the polymer.

Suitable conductive polymers for use in the present invention include polypyrrole or its derivatives, polythiophene or its derivatives, polyphenylene sulphide or its derivatives, polyaniline or its derivatives, polyindole or its derivatives, polycarbazole or its derivatives, polyacetylene or its derivatives, poly(p-phenylene vinylene) or its derivatives, as well as copolymers and/or combinations thereof. Suitable derivatives are those that contain functional groups, such as a methoxy group. Examples within the range of other optional functional groups are alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulfonyloxy, arylsulfenyloxy, heterocyclyl, heterocycloxy, heterocyclamino, haloheterocyclyl, alkylsulfenyl, arylsulfenyl, carboalkoxy, carboaryloxy, mercapto, alkylthio, benzylthio, acylthio, sulfonate, carboxylate, phosphonate and nitrate groups or combinations thereof. The hydrocarbon groups referred to in the above list are preferably 10 carbon atoms or less in length, and can be straight chained, branched or cyclic. Preferred conductive polymers for use in the method of the present invention include polythiophene and its derivatives (e.g. PEDOT), polypyrrole and its derivatives and polyaniline and its derivatives.

The conductive polymer is formed by electropolymerisation of polymeric subunits capable of polymerising to form a conductive polymer. For example, the conductive polymers PEDOT, polypyrrole and polyaniline can be formed by electropolymerisation of the monomer EDOT, pyrrole or aniline, respectively.

### Electropolymerisation of the Conductive Polymer

In the method described herein, a conductive polymer is formed within the polymeric network by electropolymerisation. Electropolymerisation is a well-known process for forming conductive polymers. Electropolymerisation of a polymer is also referred to as electrodeposition. In the present context, electropolymerisation (and electrodeposition) refers to a process of applying an electrical voltage in the form of either a current or a voltage potential to polymerise a polymer subunit, such as a monomer. In the method of the present invention, the charge promotes polymerisation of the conductive polymer from the SCCP (i.e. the site of nucleation). For example, 3,4-ethylene dioxythiophene (EDOT; a monomer suitable for forming the conductive polymer PEDOT) may be introduced into the polymer network having a SCCP dispersed in it, and by applying a charge across at least a portion of the network, polymerisation of the EDOT occurs with PEDOT:PSS (i.e. the SCCP) providing a nucleation site from which the polymer grows to form the conductive polymer.

Electropolymerisation can be performed in either potentiostatic or galvanostatic mode. In a preferred embodiment, galvanostatic electropolymerisation is used in the method described herein. The voltages and currents selected for the electropolymerisation will depend on the polymer subunit used to form the conductive polymer, the SCCP, and the polymeric network used. A person skilled in the art will be able to take account of the variables and be able to select appropriate conditions to perform the electropolymerisation. For galvanostatic electropolymerisation, suitable currents are typically from about 0.1 to 6 mA/cm². For potentiostatic electropolymerisation, suitable voltages are typically from about 1.2 to about 3 volts.

In the method disclosed herein, polymerisation starts at a nucleation site (i.e. the SCCP) and, by the process of electropolymerisation, forms a polymer which becomes the conductive polymer as the polymer chain grows.

Typically, the polymeric network is not bound to an electrode during the electropolymerisation of the conductive polymer.

Typically, the electropolymerisation of the conductive polymer comprises contacting the polymeric network with a solution of a polymer subunit capable of polymerising to form the conductive polymer, e.g. by immersing the polymeric network in the solution, and applying an electrical potential across the polymeric network.

The electropolymerisation of the conductive polymer is continued until the growth of the conductive polymer is sufficient to provide electrical conductivity to the resultant polymeric material.

Accordingly, in an embodiment, the present invention provides a method of preparing an electrically conductive polymeric material, the method comprising:
providing a polymeric network having a short chain conductive polymer (SCCP) dispersed in the polymeric network;
contacting the polymeric network with a solution of a polymer subunit of a conductive polymer, e.g. by immersing the polymeric network in the solution, and applying an electrical potential across the polymeric network to induce electropolymerisation of the conductive polymer in the polymeric network; and
continuing the electropolymerisation of the conductive polymer for a time sufficient to provide an electrically conductive polymeric material.

In some embodiments, the resultant electrically conductive polymeric material comprises the conductive polymer in an amount of from 2 to 40%, e.g. 5 to 25%, by weight based on the total weight of the dry conductive polymeric material.

### Electrically Conductive Polymeric Material

The electrically conductive polymeric material prepared by the method of the present invention may, for example, have a conductivity of greater than about 10 S/cm and/or a charge storage capacity of greater than about 10 mC/cm². In some embodiments, the electrically conductive polymeric material has a charge storage capacity of greater than about 20 mC/cm². In some embodiments, the charge storage capacity is in the range of from 20 to 300 mC/cm², e.g. 20-250, 20-200, 20-150, 50-300, 50-250, 50-200 or 50-150 mC/cm². In some embodiments, the conductivity is greater than about 5, 8, 10, 15, 20, 30, 50, 80, 100, 200 S/cm. In some embodiments, the conductivity is in the range of from 5 to 250 S/cm, e.g. 10-200 or 50-200 S/cm.

In the method disclosed herein, the electropolymerisation of the conductive polymer may be in a localised portion of the polymeric network or may be throughout the polymeric network. In some embodiments the electropolymerisation takes place in a predetermined region of the polymeric network. For example, the region may be selected by any one or more of (i) introducing the SCCP into only a predetermined region of the polymeric network; (ii) introducing the polymer subunit from which the conductive polymer is formed into only a predetermined region of the polymeric network; or (iii) applying the electropolymerisation charge to only a predetermined region of the polymeric network (for example, by use of patterned or shaped electrodes to apply the charge). Other methods may also be used to electropolymerise the conductive polymer in only predetermined regions of the polymeric network.

The method of the present invention can be used to prepare soft, flexible conductive materials. The method enables the preparation of materials having fast charge transfer and high charge injection capability, beyond that offered by conventional conductive polymer loaded materials.

By localising the SCCP within localised regions of a bulk non-conducting polymeric material, the method of the present invention enables the preparation of a product comprising a conductive component comprising a conductive polymer localised to specific areas within the bulk non-conductive polymeric material. This enables the fabrication of freestanding soft polymer based electrode arrays and biosensors that are not associated with an underlying metallic array.

The SCCP can be localised within regions of a bulk non-conductive polymeric material by a variety of techniques. For example, a substrate formed of a bulk non-conductive polymer may be formed having a pattern of spaces on the surface or within the substrate. The spaces may be formed by the use of a mould, 3D printing, 3D lithography or other techniques. A mixture for forming the polymeric material having dispersed therein a SCCP may be placed in these spaces and the polymeric material formed. Following electropolymerisation of the conductive polymer, the conductive polymer will be located in the regions which contained the SCCP.

The following examples describe the formation of electrode tracks in hydrogel constructs. Similar principles can also be applied to elastomers. The method enables the construction of fully flexible and robust electronics which do not contain inorganic and/or rigid metallic elements.

Products comprising an electrically conductive polymeric material prepared by the method of the present invention can be used for a range of bioelectronic devices, from sensors and diagnostics to stimulators (both external and implantable). The electrical properties of the conductive tracks and electrodes enable improvements in both stimulation capacity and sensitivity of recordings. Electrically conductive polymeric materials prepared by the method of the present invention may be used in products including, but are not limited to, cochlear implants, cardiac pacemakers, deep brain stimulators (where flexibility is a major limitation that causes device failure), urinary pacemakers (both implanted and externally applied), wound healing, non-invasive neural mapping, glucose and other biosensors.

The electrically conductive polymeric materials prepared by the method of the present invention may have electrical properties on the order of 10× better than a standard metal array of the same size.

The method of the present invention enables the preparation of electrically conductive polymeric materials that are not bound to an inorganic surface such as a metal surface. The electrically conductive polymeric materials prepared by the method of the present invention may have a variety of shapes.

In one aspect, the present invention provides a free standing flexible electrically conductive polymeric material comprising a conductive polymer within a polymeric network, wherein the electrically conductive polymeric material has a conductivity of greater than about 10 S/cm. In some embodiments, the conductivity is in the range of from 5 to 250 S/cm, e.g. 10-200 or 50-200 S/cm. In another aspect, the present invention provides a free standing flexible electrically conductive polymeric material comprising a conductive polymer within a polymeric network, wherein the electrically conductive polymeric material has a charge storage capacity of greater than about 10 mC/cm². In some embodiments, the charge storage capacity is in the range of from 20 to 300 mC/cm², e.g. 20-250, 20-200, 20-150, 50-300, 50-250, 50-200, 50-150 mC/cm².

In one aspect, the present invention provides an electrically conductive polymeric material having a dimension of greater than about 200 μm in all directions. The electrically conductive polymeric material may be non-laminar or non-planar in shape. In one embodiment, the electrically conductive polymeric material comprises a conductive polymer substantially homogeneously distributed throughout the polymeric material. In one embodiment, the electrically conductive polymeric material is not bound to an inorganic surface.

In another aspect, the present invention provides an electrically conductive polymeric material having a charge injection limit of more than 300 μC/cm², wherein polymeric material it is not bound to an inorganic surface (i.e. the polymeric material is a freestanding polymeric material).

In another aspect, the present invention provides a polymeric material comprising one or more regions which are electrically conductive and one or more regions which are non-conductive, wherein the conductive regions and non-conductive regions are integrally bound to each other and wherein at least one of the electrically conductive regions has a dimension of greater than about 200 μm in all directions.

### EXAMPLES

The present invention is further described below by reference to the following non-limiting Examples.

### Example 1

Comparison of Primary and Secondary Nucleation for Electrochemical Polymerisation of Conductive Polymers within Poly(Vinyl Alcohol) Methacrylate (PVA-MA) Hydrogels

A comparison of two potential methods for introducing nucleation into hydrogels, (i) primary heterogeneous and (ii) secondary nucleation, are presented. Specifically, the introduction of (i) conductive bulk metallic glass (BMG) particles, composed of Mg₆₄Zn₃₀Ca₅Na₁, and (ii) a dispersion of chemically synthesised poly(3,4-ethylene dioxythiophene)-poly(styrene sulfonate) (PEDOT:PSS). The BMG particles were chosen as conductive particles which could be removed from the material post-polymerisation by acidic degradation. PEDOT:PSS in this example was provided as an aqueous dispersion of small chain length polymer chains, and is only minimally conductive.

Both BMG and PEDOT:PSS systems were loaded into poly(vinyl alcohol) (PVA) hydrogel at varied percentage content and the conductive polymer (CP) poly(3,4-ethylene dioxythiophene) (PEDOT) was electropolymerised (electrodeposited) through the PVA. The electrical properties and physical appearance of the gels were analysed at time points between 10 and 80 mins of electropolymerisation to determine and compare the extent of PEDOT polymerisation within the PVA.

(i) Primary Nucleation: BMG Particles

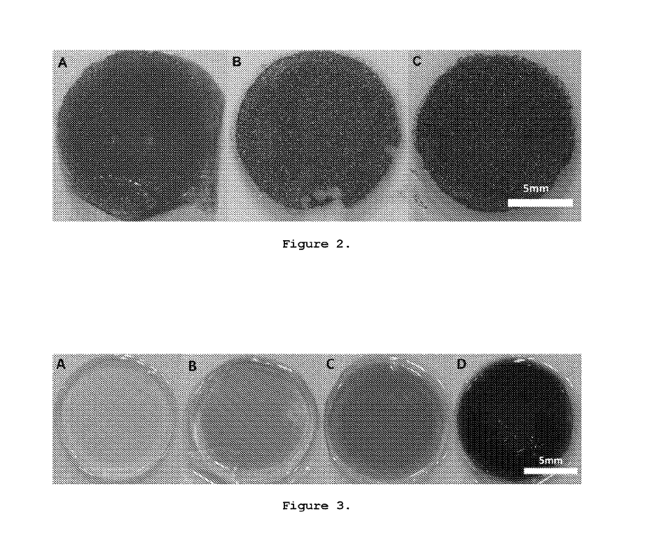

BMG particles were ground into fine particles and passed through a 45 μm sieve. The particles were then loaded at 5, 10 and 15 wt % in an 18 wt % aqueous solution of methacrylated PVA (PVA-MA, with 4 functional groups per chain) with a 2 wt % methacrylated heparin component (PVA-Hep). The heparin component dopes the PEDOT and further supports growth of the CP within the hydrogel (Poole-Warren L. et al. Expert Rev Med Devices. 2010; 7(1):35-49). The BMG loaded gels were cross-linked by photopolymerisation for 180 s in the presence of a photoinitiator (0.1 wt % 12959 and 30 mW UV light). Despite increasing BMG concentration, the hydrogels produced did not have a clear difference in appearance, as shown in FIG. 2. However, the 15 wt % BMG loaded hydrogels were observed to have a different consistency to the hydrogel discs with lower particle loadings, appearing tacky and soft in the centre. To achieve adequate crosslinking at this higher loading both sides of the discs were exposed to UV light. It is proposed that at the higher loading BMG particles caused reduced penetration of light within the hydrogel and impeded cross-linking. Another observation was that bubble formation occurred within hydrogels at higher loadings of BMG.

(ii) Secondary Nucleation: PEDOT:PSS (a Short Chain Conductive Polymer)

PEDOT:PSS (Orgacon™, Sigma-Aldrich, Cat#739332) was dispersed within a 20 wt % PVA-MA macromer solution at 0.01, 0.05, 0.1 and 0.5 wt %. In this system, heparin was not added as the PSS chain which is covalently bound to each PEDOT chain in the dispersion provides doping through the sulfonate groups. These hydrogels were crosslinked by photopolymerisation for 180 s under equivalent conditions to those above. FIG. 3 shows an increasing blue coloration with increasing concentration of PEDOT:PSS loaded into the hydrogels. At the lower loadings of 0.01 wt % and 0.05 wt % (FIGS. 3A and B), there was evidence of phase separation, with the PEDOT:PSS dispersion appearing to coalesce at the centre of the hydrogel disc, leaving a clear border region at the edge.

Analysis of Hydrogels Containing BMC and PEDOT:PSS Prior to Electropolymerisation

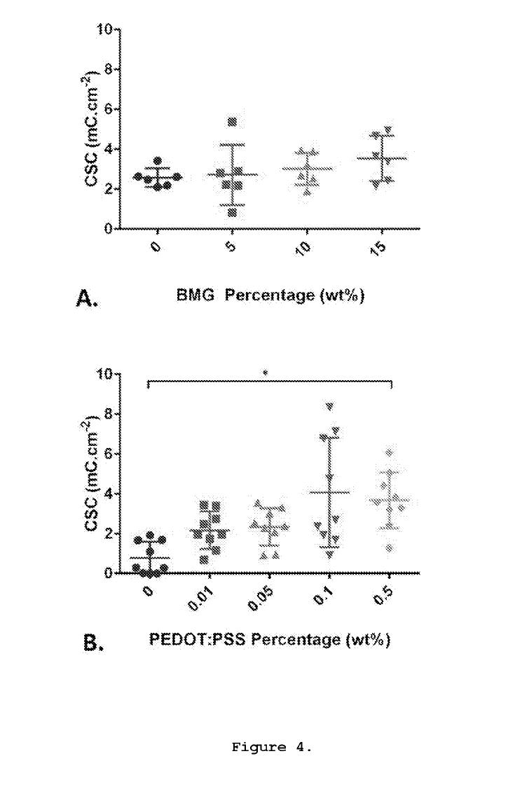

Prior to attempting electropolymerisation of conductive polymer in the hydrogels, the electrical properties of the gels were analysed to determine the degree of electrochemical conductivity imparted by BMG and PEDOT:PSS inclusions. In both of systems it was not expected that the inclusions would impart conductivity unless a percolation threshold, where a continuous path of conductive material is created within the non-conductive PVA, was reached. It can be seen in FIG. 4A that for the BMG materials there was no increase in charge storage capacity (CSC) measured by cyclic voltammetry (CV) in phosphate buffered saline (PBS), with the voltage ramped between −600 mV and +800 mV at 150 mV/s. For the PEDOT:PSS loaded PVA (FIG. 4B) a small increase in the baseline CSC was seen when the loading was increased to 0.5 wt %. The increase in CSC likely occurs only when the PEDOT:PSS chains are closely associated and form an electrical path from the underlying electrode through the PVA volume.

Electropolymerisation

Electropolymerisation of PEDOT was performed as described previously (Green R A et al. Bioactive Conducting Polymers for Neural Interfaces Application to Vision Prosthesis. 2009; (Cv):60-63, the contents of which is incorporated herein by reference). Briefly, electrochemical growth of PEDOT was conducted using a potentiostat in two electrode galvanostatic mode (eDAQ, Australia). The aqueous EDOT solution was produced at 0.03M and deposition conducted at 0.5 mA/cm² with the hydrogel overlying an ITO working electrode with a large platinum counter electrode. Electropolymerisation was conducted in 10 min intervals with CV and electrochemical impedance spectroscopy (EIS) performed following 10, 20, 40 and 80 mins of polymerisation in PBS. EIS is a frequency dependant measurement where impedance and phase are reported together to provide details of both the resistive and capacitive behaviour of the material. The impedance magnitude is the attenuation of the magnitude of the signal, whereas phase angle is the time shift between applied voltage and measured current. CV on the other hand measures the electrochemical response of the material, which can be used to determine its charge storage capacity. Since PEDOT is an optically opaque dark blue polymer, light microscopy images were also obtained to examine the physical growth of the PEDOT through the transparent PVA.

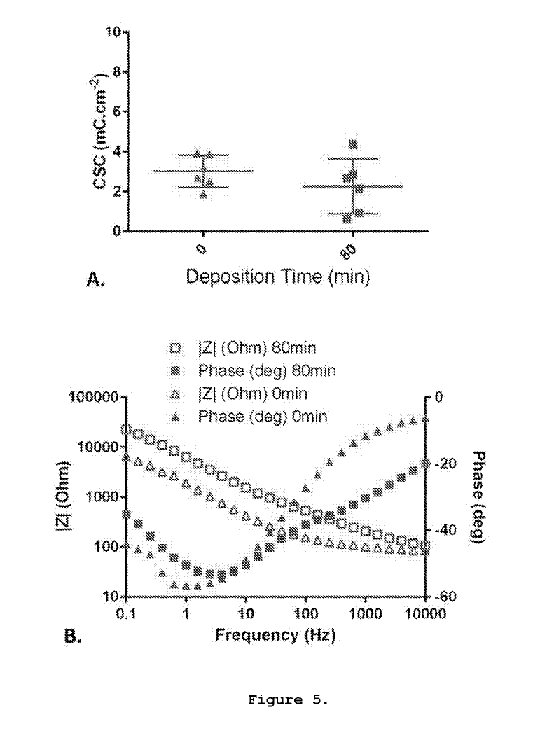

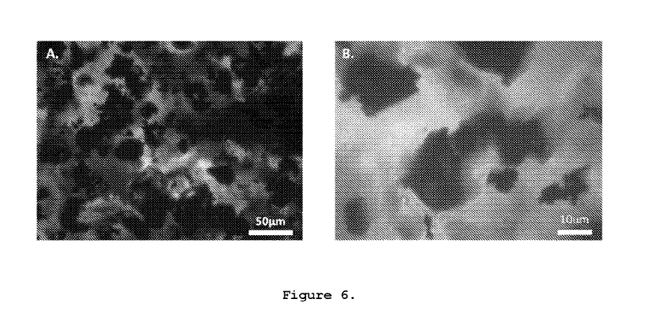

For the BMG loaded samples there was no evidence of PEDOT growth throughout the PVA-Hep hydrogel. The electrical characterisation shown in FIG. 5 demonstrates that despite application of charge for up to 80 mins, there was no increase in CSC or decrease in impedance, as would be expected from the growth of CP chains. Although the data for only the 10 wt % BMG loaded PVA-Hep is shown, the same results were observed for all BMG loadings. Supporting this finding is the light microscope (Olympus CKX41) images in FIG. 6, which confirm that there is no growth of the expected blue, opaque electrodeposited PEDOT. The high magnification image also reveals the degree of particle separation of BMG within the hydrogel matrix. It is expected that this was a limiting factor as the relatively large particles were too dispersed to enable a percolation threshold to be achieved, imparting a low voltage and hence low energy path from which PEDOT growth could nucleate.

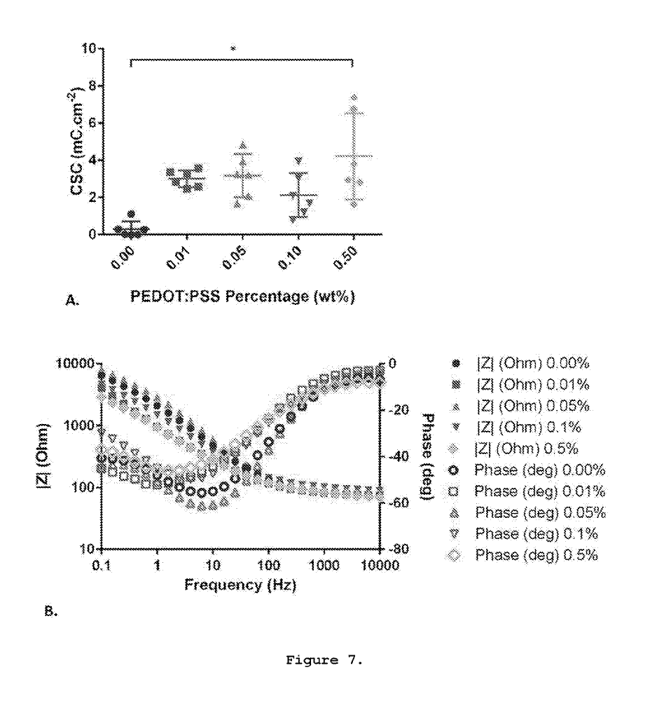

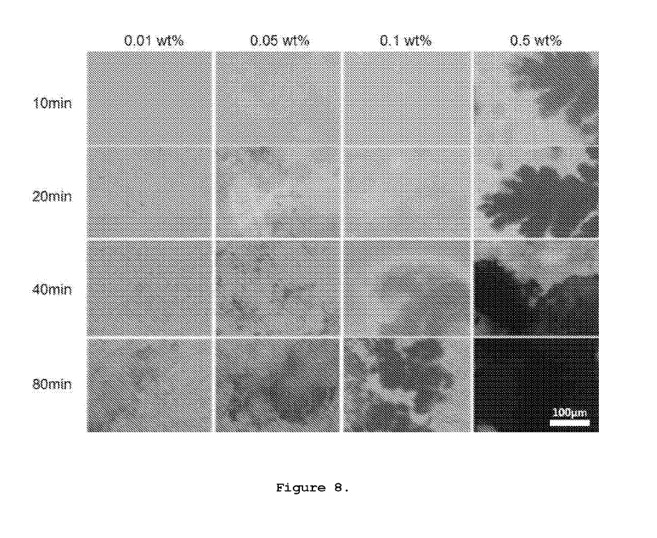

In the samples loaded with PEDOT:PSS at 0.5 wt %, both electrical characterisation and physical imaging indicated growth of electrodeposited PEDOT. FIG. 7 shows the CSC and impedance properties for the PEDOT:PSS loaded hydrogel after electropolymerisation of PEDOT. Following 80 mins of electropolymerisation there was no change in the average CSC or impedance of the 0.01, 0.05 and 0.1 wt % loaded PVA, but some increase in electroactivity observed for the 0.5 wt % loadings. The optical micrographs concur with these results, as shown in FIG. 8 where only the 0.5 wt % PEDOT:PSS loaded PVA showed substantial growth of PEDOT across the 80 min electropolymerisation.

Nucleation of PEDOT through the PEDOT:PSS loaded gels was observed at highest loading after only 10 minutes of polymerisation. However, there was no increase in electrochemical activity, most likely due to the conductive pathway being incomplete through the material. Essentially, the PEDOT chain length increases from the initial nucleation sites (the PEDOT:PSS chains), but each nucleation site increases in isolation of other nucleation sites. Since the CV and EIS analyses rely on electrical contact with the underlying working electrode (in this case a stainless steel base); the increasing PEDOT volume in the hydrogel will not be measurable until the network is fully connected or at least until the growth of the PEDOT extends to the base where the disc contacts the working electrode. For this reason the data shows an “on/off” conductive phenomenon in which the gels are either electroactive or they are not. While there were only small, not statistically significant increases in electroactivity for the 0.5 wt % PEDOT:PSS loaded PVA over the 80 min electropolymerisation, the optical micrographs clearly demonstrate that there is an increasing amount of PEDOT within the hydrogel. It was also evident (FIG. 8) that growth of PEDOT within the 0.1 wt % PEDOT:PSS loaded PVA occurred, however this did not contribute to the electrochemical performance of the material as measured using this technique. In the samples with lower loadings of 0.01 and 0.05 wt %, there was evidence of PEDOT forming at 40 and 80 mins, but this was sparse and was observed to be mainly associated with the surface contacting the working electrode. To further understand how the growth of PEDOT within the PVA was affecting the electroactivity, electropolymerisation was continued for a further 80 mins (160 min total) for the 0.1 wt % and 0.5 wt % PEDOT:PSS loaded PVA samples.

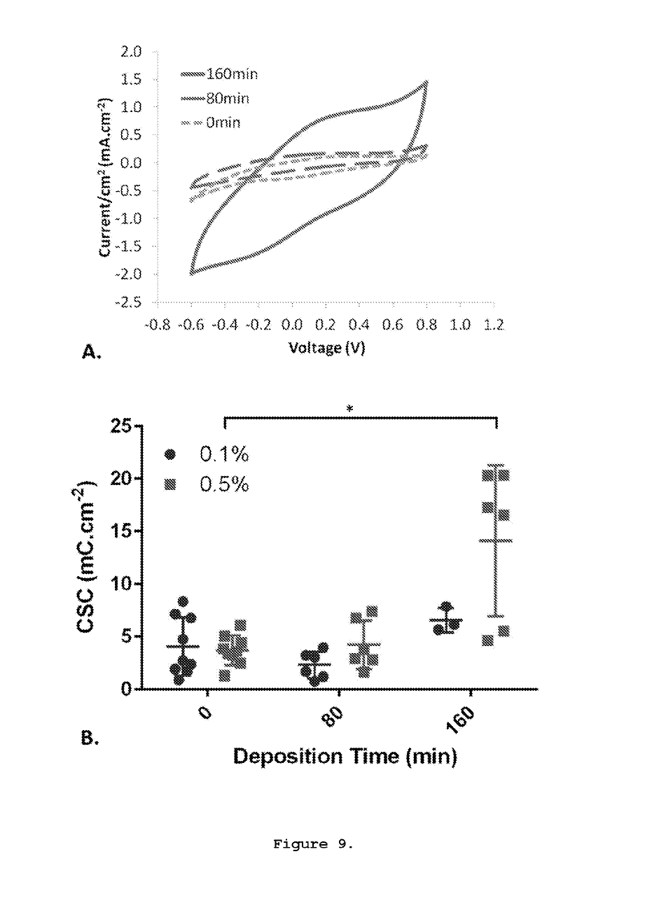

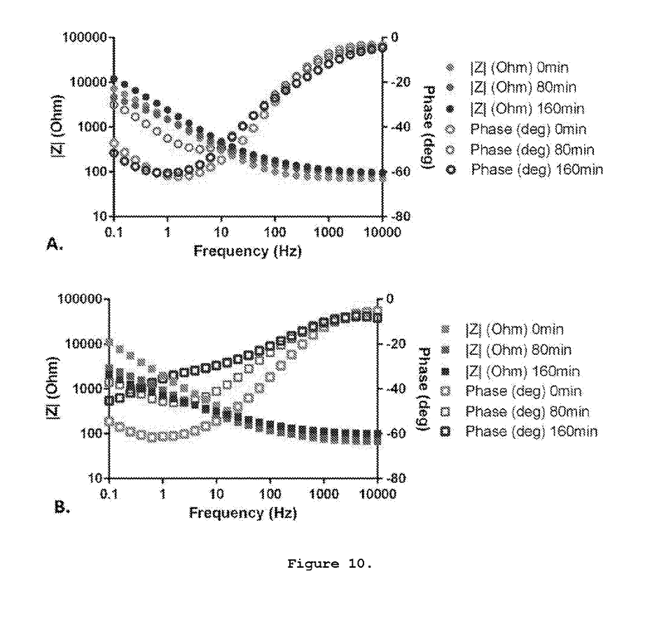

The cyclic voltammetry curves in FIG. 9A for the 0.5 wt % PEDOT:PSS loaded PVA show that there was minimal change in electroactivity for the first 80 mins, but a significant increase as electropolymerisation was continued for 160 mins. This is quantitated in the CSC generated from these curves, shown in FIG. 9B for the 0.1 wt % and 0.5 wt % PEDOT:PSS loaded PVA. It is clear that there was an increase in electroactivity associated with PEDOT electropolymerisation for the 0.5 wt % PEDOT:PSS loaded PVA with average CSC varying from 3.8 mC/cm² at 0 min to 16 mC/cm² at 160 min. However, in the 0.1 wt % PEDOT:PSS loaded PVA the average CSC ranged from 4.1 mC/cm² at 0 min to 6.6 mC/cm² at 160 min, suggesting that some growth of the CP occurred, but the network had not yet connected sufficiently to produce a highly electroactive material. In FIG. 10, it can be seen that there were statistically significant changes in the electrochemical impedance for the 0.5 wt % PEDOT:PSS loaded gels. The average phase lag at 1 Hz was decreased from 61.2° to 35.4° with the average impedance decreasing in parallel from 1990Ω to 715Ω. This behaviour was not seen in the 0.1 wt % PEDOT:PSS loaded gels, which had clearly not developed a sufficient amount of electrodeposited PEDOT to enable detectable differences in the electrochemical properties. The improvement in electroactivity at 0.5 wt % PEDOT:PSS suggests that the growth of PEDOT nodules or clouds within the PVA continues until the isolated particles connect, enabling measurement of the PEDOT network electrical properties. However, this analysis method is clearly limited, and alternate methods, such as DC four-point probe conductivity, may prove more effective in analysis.

Results Summary

The electrochemical analyses of the loaded hydrogels prior to and following electropolymerisation demonstrates that there are several factors which influence nucleation and PEDOT growth. The particles in the BMG loaded PVA-Hep did not impart electrochemical conductivity, even at high loading. It was observed when depositing PEDOT through the BMG loaded gels that depositions tended to take place on the working electrode, beneath the hydrogel disc. As such, one can conclude that in this system the working electrode is a preferential energy cost site for nucleation to occur rather than through the hydrogel and the foreign particles do not provide a point of low potential for PEDOT precipitation and growth. This is most likely because, as isolated particles within an insulative material, they were not part of the electrical circuit. In the PEDOT:PSS system, nucleation was more readily observed at the higher polymer loadings where electrochemical conductivity was present in the PVA (although in a small amount) prior to electropolymerisation of the PEDOT.

### Example 2

As discussed above, nucleation of a CP within a polymeric network can occur through either primary or secondary mechanisms. Primary nucleation occurs where there is no existing CP and at the site where the Gibbs free energy is the lowest. Secondary nucleation is the new growth of a CP from an existing CP chain. This is also the site of lowest energy.

As shown in Example 1, secondary nucleation sites can be provided within a hydrogel that facilitate subsequent growth of a CP within that volume. This example demonstrates that this technique can be used to pattern conductive tracks within non-conductive hydrogels.

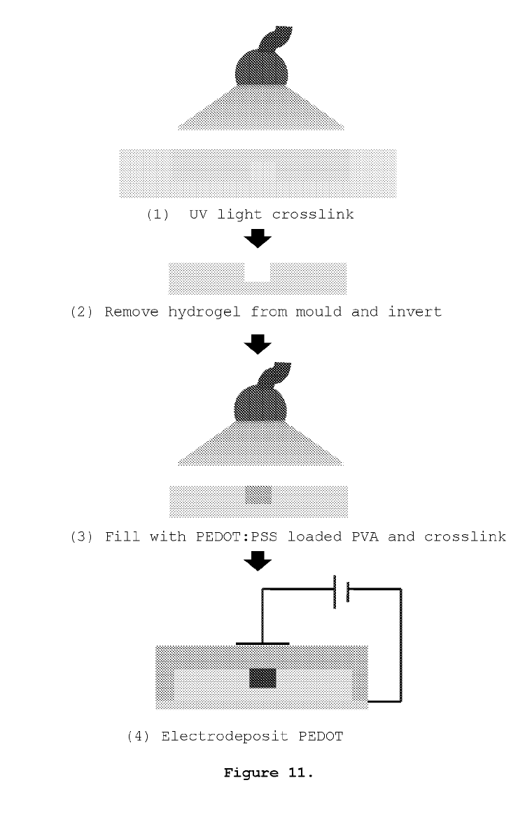

To demonstrate the fabrication of a conductive hydrogel (CH) track within a non-conductive hydrogel, a silicone rubber mould was fabricated. This enabled the formation of a 5 mm diameter hydrogel disc with a negative imprint of a 1×1 mm square track across the center. PVA was crosslinked under UV light to form the non-conductive hydrogel bulk of the sample. Subsequently, PVA (loaded with PEDOT:PSS) was cross-linked within the track negative to create the patterned area where subsequent electropolymerisation of PEDOT was required. The process is shown schematically in FIG. 11. The resulting construct was characterized electrically and cell compatibility with materials was assessed.

### Methodology

A. Fabrication of Hydrogel Electrode Tracks

Large disc samples were produced with 5 mm diameter at 1.5 mm thick. A non-degradable and not conductive hydrogel was formed from a macromer solution of 20 wt % methacrylate modified PVA (A. Nilasaroya et al. Biomaterials, vol. 29, pp. 4658-4664, 2008). The hydrogel film was crosslinked with ultra-violet (UV) light (30 mW/cm², 365 nm) for 180 s in a silicone rubber mold which created an 1×1 mm channel within the disc. This embossed channel was then filled with a macromer solution of 18 wt % PVA and 2 wt % heparin loaded with a dispersion of chemically synthesized CP being poly(3,4-ethylene dioxythiophene)-poly(styrene sulfonate) (PEDOT:PSS; Orgacon™, Sigma-Aldrich, Cat#739332) at 0.5 wt %. The construct was then exposed to UV light for a further 180 s to create a track. PEDOT was deposited through this gel from an aqueous solution of 0.03 M EDOT at 0.5 mA/cm² for up to 20 min. The charge required for electropolymerization was applied using an indium tin oxide (ITO) slide on which the sample was placed. A 200 μl droplet of the EDOT solution was placed over the sample and a large Pt counter electrode was brought into contact with the fluid. Charge was applied in 10 min increments with the EDOT solution was replaced following the first deposition period. Electrical measurements and optical imaging were conducted before and after PEDOT electropolymerization.

B. Electrical Properties

Cyclic voltammetry (CV) was used to characterize the electrochemical activity of constructs at each stage of fabrication. A three electrode cell was formed by placing the construct on a stainless steel (SS) base plate. The area through which the charge was transferred was restricted by placing a silicone gasket over the sample surface to expose only the CH track area to the phosphate buffered saline (PBS) electrolyte solution. Measurements were made via a large Pt counter electrode and an isolated Ag/AgCl reference electrode. Voltage was cycled between −600 and 800 mV at 150 mV/s for 20 cycles using an eDaq potentiostat and eCorder unit (eDaq, Aust). The charge storage capacity (CSC) was calculated by integrating the resulting current waveform relative to time. A measurement was made of the construct before patterning of the track (Stage 2 in FIG. 11), to assess the contribution to the signal of the underlying SS baseplate and PVA hydrogel.

The frequency dependent impedance spectroscopy was determined using an eDaq impedance analyzer. The same 3 electrode cell was used to record the impedance of samples exposed to 50 mV sinusoids delivered from 1 Hz to 10 kHz.

C. HL-1 Cell Compatibility

A clonal line of excitable cells obtained from cardiac muscle, known as HL-1s, were cultured directly onto the constructs. A tissue culture plastic (TCP) well plate was used as a control. Cells were plated at 1×10⁵ cells/c² in Claycomb Medium supplemented with 10% fetal bovine serum (FBS), 1% penicillin/streptomycin, 0.1 mM norepinephrine and 2 mM L-glutamine. Cells were imaged at 48 hr by light microscopy.

### Results

A. Fabrication of Hydrogel Electrode Tracks

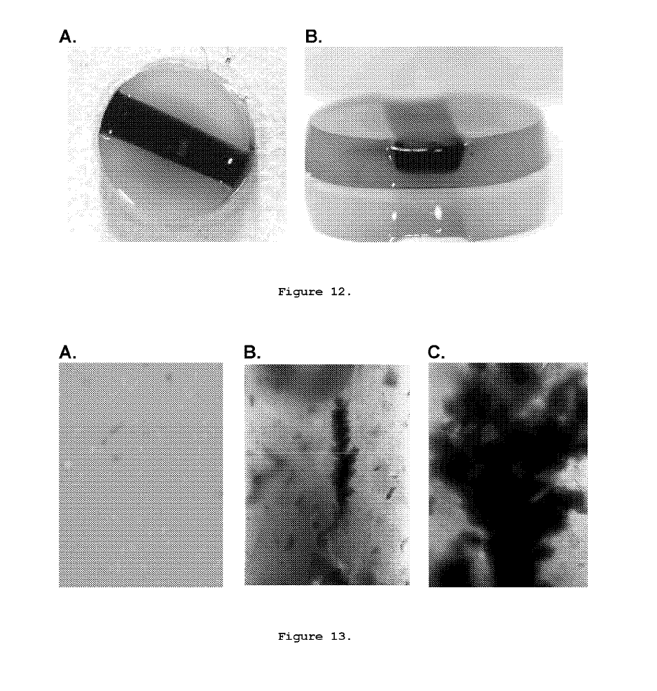

The construct was fabricated as a hydrated disc which a clearly delineated track at the center, as shown in FIG. 12. While the samples were made from a 5 mm diameter circular mold with a 1 mm wide track, the swelling property of the hydrogel increased these dimensions by an average of 27±3% when stored in water for a period of 18 hours. Following this initial period the dimensions were stable and unaffected by the subsequent electropolymerisation.

The electrochemical growth of PEDOT within the track was observed using light microscopy and showed that nucleation of the CP occurred and was restricted to the track area which was pre-loaded with PEDOT:PSS. CP growth was recognized by the appearance and increasing volume of opaque and dense dark blue nodules, exhibiting morphology typical of PEDOT (R. A. Green et al. Biomaterials, vol. 29, pp. 3393-3399, 2008.). The growth of the polymer at 0, 10 and 20 min is shown in FIG. 13. It was also noted that a small amount of dark blue powder was formed on the ITO glass where the EDOT solution was in contact with both the working and counter electrode. However, these particulates were only loosely aggregated and were washed away from both the sample and ITO with DI water at the termination of electropolymerisation.

B. Electrical Properties

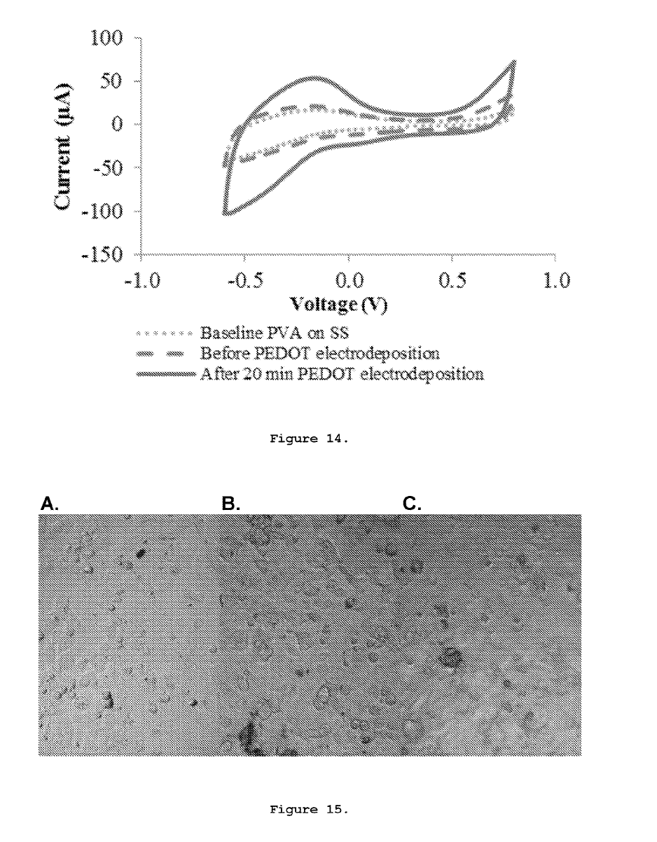

The CSC of the model electrode track was measured at each stage of fabrication by cyclic voltammetry. The growth of the PEDOT within the CH was evidenced by an increase in CSC from 3.2 mC/cm² before electropolymerisation, up to 7.1 mC/cm^{z} following 20 min of electropolymerisation. The hysteresis loop created by the CV is shown in FIG. 14. It is important to note that the shape of the curve is influenced by the underlying SS electrode and the large area of PVA through which the current is transferred before reaching the electrolyte in which the reference electrode is located above the track surface.

The electrochemical impedance spectroscopy results supported the voltammetry findings, indicating an increased charge transfer capacity as the PEDOT growth is continued. This was evidenced by an average reduction in impedance and the phase lag at low frequency was shifted, being reduced by an average of 9.6° at 100 Hz.

C. HL-1 Cell Compatibility

The HL-1 cardiomyocyte cell line was found to attach to the constructs and proliferated over a period of 48 hr. There was no visible difference in the cell numbers before or after the electropolymerisation of the PEDOT (shown in FIG. 15). Additionally, the cells did not appear to preference the track or PVA region of the construct. The cells on the TCP control had a more flattened morphology than those on the hydrogel substrates.

### Discussion

It has been shown in Example 1 that SCCPs included within a hydrogel provide nucleation for CP growth. The growth of PEDOT within patterned tracks can be controlled through the provision of SCCPs and the parameters used for subsequent electropolymerisation of PEDOT. This technique can be used to create patterned hydrogel constructs with areas of high electroactivity and may be advantageous for producing scalable, soft, organic implantable electronics.

The formation of PEDOT within the tracks containing the precursor CP was visually observed. At 10 min only small, relatively isolated areas of PEDOT were seen. As the electropolymerisation was continued the PEDOT nodules increased in size, filling the hydrogel volume. It is believed that the provision of both the precursor PEDOT:PSS chains and the heparin molecule, which has been shown to dope CPs in CH coating constructs, is advantageous in selectively controlling the formation of the PEDOT within the track volume. Growth was not uniform across the track, but extended to the border region of the homogenous PVA. Since the adjacent PVA only hydrogel did not contain either the PEDOT:PSS precursor chains or an available dopant molecule, PEDOT did not extend into this region. This technique provides advantages over the prior art in the development of hydrogel electronics. In studies by Sekine et al. (Journal of the American Chemical Society, vol. 132, pp. 13174-13175, 2010) CP tracks were grown on a patterned ITO surface and then embedded in an agarose hydrogel. The whole construct was then removed from the ITO by electroactuation. It was found that while these tracks had good electroactivity, they suffered from mechanical failure upon flexing as the CP component was friable and stiff. CHs have been shown in the prior art to have improved mechanical properties over homogeneous CPs, and it is believed this characteristic will improve the robustness of the overall construct while simultaneously reducing the stiffness.

This Example shows that the electropolymerisation of PEDOT was associated with an increase in charge transfer capacity and a decrease in impedance. Additionally, it should be noted that the suspension of PEDOT:PSS within the PVA did not impart a significant increase in electroactivity of the construct. These results concurred with Example 1 on unpatterned hydrogel discs, however, in this Example, the electrochemical benefit obtained from CP growth was realized at earlier time points. It is believed that this is due to the inclusion of heparin within the PVA-PEDOT:PSS hydrogel track. In Example 1, the only dopant available was the excess of PSS on the PEDOT:PSS copolymer suspension. As a result, electrochemical improvements were not realized until 80 min of PEDOT electrochemical deposition. The extra dopant incorporated by inclusion of heparin appears to expedite the electrochemical growth of PEDOT.

In the electrochemical cell used to generate these results, the charge was passed through the underlying PVA hydrogel prior to passing through the track. This enabled the characterization of PEDOT growth as a function of the entire construct, but as seen in FIG. 14, the curve generated had features typical of both PEDOT and the underlying SS used to apply the electrical potential. It is believed that measurements of DC conductivity would be useful in assessing the potential of these tracks in carrying charge for electronic devices.

This example also shows that these constructs are compatible with a cell line derived from cardiomyocytes. The cells adhered to both the PVA and CH track with similar morphologies to that of TCP controls. This study demonstrates cell compatibility. In addition, stem cell differentiation into neural or cardiac lineages may be able to be controlled through provision of an electroactive substrate (K. H. Lie et al. Human Embryonic Stem. Cells Handbook. vol. 873, K. Turksen, Ed., ed USA: Springer, Humana Press, 2012, pp. 237-246), and as such, these constructs may provide an assessment tool for stem cell differentiation as a function of both the electroactivity and substrate stiffness.

### Conclusion

This example demonstrates that electrochemical nucleation of CP growth within a non-conductive material can be tailored to create patterned areas of significant conductivity. This technique provides a method for the development of soft, freestanding bioelectronics. Loading PVA with 0.5 wt % PEDOT:PSS enabled the fabrication of a free-standing, electroactive construct following PEDOT electropolymerisation.

### Example 3

### Soft and Flexible Electroactive Materials for Neuroprosthetic Devices

In this study, the conductive polymer complex poly(3,4-ethylenedioxythiophene:polystyrenesulfonate (PEDOT:PSS) and polyurethane (PU) were used to fabricate conductive elastomers (CEs). In order to fabricate the PU-PEDOT:PSS elastomer, various loadings of PEDOT:PSS were dispersed in a solution of PU dissolved in dimethylsulfoxide. The solution was cast and dried to produce a film comprising PEDOT:PSS in an amount of 1, 4, 8, 16 or 24 wt %. Electropolymerisation of PEDOT within the film was then performed in a similar manner to that described in Example 1 but using a solution of EDOT in dimethylsulfoxide. Cyclic voltammetry (CV) was used to assess the charge storage capacity (CSC) of the films prior to electropolymerisation and after electropolymerisation. The films after 40 minutes of electropolymerisation of PEDOT demonstrated a greater than 3 times increase in charge storage capacity compared to the films prior to electropolymerisation of PEDOT. The resultant conductive films were soft, flexible and had good tensile strength.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method of preparing an electrically conductive polymeric material, the method comprising:
providing a polymeric network having a short chain conductive polymer (SCCP) dispersed in the polymeric network;
electropolymerising a conductive polymer (CP) within the polymeric network.

2. The method according to claim 1, wherein the polymeric network is a hydrogel.

3. The method according to claim 1, wherein the polymeric network is an elastomer.

4. The method according to claim 1, wherein the polymeric network, prior to electropolymerisation of the conductive polymer, is non-conductive.

5. The method according to claim 1, wherein the short chain conductive polymer comprises from about 5 to about 1000 monomeric units.

6. The method according to claim 1, wherein the short chain conductive polymer is PEDOT:PSS or tetramethacrylate poly(3,4-ethylene dioxythiophene).

7. The method according to claim 1, wherein the conductive polymer is PEDOT, polypyrrole or polyaniline.

8. The method according to claim 1, wherein electropolymerisation of the conductive polymer comprises:
contacting the polymeric network with a solution comprising monomer of the conductive polymer; and
applying an electrical potential across the polymeric network.

9. The method according to claim 1, wherein the polymeric network having a SCCP dispersed in the network comprises a localised region of a polymeric material.

10. The method according to claim 1, wherein the electrically conductive polymeric material has a conductivity of greater than about 10 S/cm.

11. The method according to claim 1, wherein the electrically conductive polymeric material has a charge storage capacity of greater than about 10 mC/cm2.

12. A device comprising an electrically conductive polymeric material prepared by the method of claim 1.

13. A free standing flexible electrically conductive polymeric material comprising a conductive polymer within a polymeric network.

14. The free standing flexible electrically conductive polymeric material according to claim 13, wherein the polymeric network is a hydrogel.

15. The free standing flexible electrically conductive polymeric material according to claim 13, wherein the polymeric network is an elastomer.

16. The free standing flexible electrically conductive polymeric material according to claim 13, wherein the conductive polymer is PEDOT, polypyrrole or polyaniline.

17. The free standing flexible electrically conductive polymeric material according to claim 13, wherein the conductive polymeric material has a charge injection limit of more than 300 μC/cm2.

18. The free standing flexible electrically conductive polymeric material according to claim 13, wherein the conductive polymeric material has a dimension of greater than 200 μm in all directions.

19. A polymeric material comprising one or more regions which are electrically conductive and one or more regions which are non-conductive, wherein the conductive regions and non-conductive regions are integrally bound to each other and wherein at least one of the electrically conductive regions has a dimension of greater than about 200 μm in all directions.
